(19) United States
(12) Patent Application Publication
(10) Pub. No.: US 2017/0306105 A1
(43) Pub. Date: Oct. 26, 2017

(54) BATCH MIXING PROCESS FOR ETHYLCELLULOSE POLYMER DISPERSIONS

(71) Applicant: Dow Global Technologies LLC, Midland, MI (US)

(72) Inventors: David L. Malotky, Midland, MI (US); Susan L. Jordan, Collegeville, PA (US)

(21) Appl. No.: 15/513,199

(22) PCT Filed: Oct. 14, 2015
(86) PCT No.: PCT/US2015/055473

§ 371 Date: Mar. 22, 2017

Related U.S. Application Data
(60) Provisional application No. 62/064,003, filed on Oct. 15, 2014.

(51) Int. Cl. C08J 3/205; B01F 3/12; A61K 47/38; B01F 7/00; B01F 7/24

(52) U.S. Cl. CPC C08J 3/2056; B01F 7/00441; B01F 7/241; A61K 47/38; B01F 3/1221; B01F 3/1214; C08J 2301/28

(57) ABSTRACT

Provided is method of making an aqueous composition comprising (a) providing a mixer comprising a sealable volume and one or more rotors inside said sealable volume; (b) placing into said sealable volume ingredients comprising ethylcellulose polymer and fatty acid; (c) placing into said sealable volume ingredients comprising water and a water-soluble base; (d) sealing said sealable volume after said steps (b) and (c); (e) then rotating one or more of said rotors while said ingredients are at a temperature above the softening point of said ethylcellulose polymer, to produce said aqueous composition; wherein said step (e) is conducted so that 90% or less of the volume of said ingredients is uncontacted by one or more of said rotors.

It is often desirable to make films that contain ethylcellulose polymer. Such films are useful, for example, as coatings applied to other films or to beads. In some cases, a collection of beads contains a drug, and each of those beads is then coated with a film that contains ethylcellulose polymer. The film that contains ethylcellulose polymer can provide controlled release of the drug when the beads are placed in an aqueous environment such as that which can be found in the human body. It is also desirable that the film have good mechanical properties such as high tensile strength, high tensile elongation, and/or surface smoothness. In the past, a common method of making such a film was to contact the beads with a solution in which ethylcellulose polymer was dissolved in an organic solvent. Organic solvents are undesirable because of environmental and health effects. It is desired to provide an aqueous coating composition that contains ethylcellulose polymer and that is capable of producing high quality films. One desirable form of such an aqueous coating composition is an aqueous ethylcellulose polymer dispersion, which is a form in which particles of ethylcellulose polymer are dispersed in a continuous aqueous medium.

Some of the previously known procedures for making such aqueous compositions are continuous methods such as methods that use extruders. Continuous methods typically suffer from one or more of the following drawbacks: continuous methods are generally not well suited for producing relatively small volumes of material, and it is generally difficult with continuous methods to make frequent changes to the components of the composition. U.S. Pat. No. 4,502,888 describes methods of making dispersions of water-insoluble polymers that employ fatty acid salts as plasticizers/stabilizers. It is desired to provide a batch method that avoids one or more of the drawbacks of continuous methods. It is also desired to provide a batch method that is capable of producing an aqueous ethylcellulose polymer dispersion having desirably small particle size.

The following is a statement of the invention.

The first aspect of the present invention is a method of making an aqueous composition comprising
(a) providing a mixer comprising a sealable volume and one or more rotors inside said sealable volume;
(b) placing into said sealable volume ingredients comprising ethylcellulose polymer and fatty acid;
(c) placing into said sealable volume ingredients comprising water and a water-soluble base;
(d) sealing said sealable volume after said steps (b) and (c);
(e) then rotating one or more of said rotors while said ingredients are at a temperature above the softening point of said ethylcellulose polymer, to produce said aqueous composition;
   wherein said step (e) is conducted so that 90% or less of the volume of said ingredients is uncontacted by one or more of said rotors;
   wherein said aqueous composition comprises particles dispersed in an aqueous medium, wherein said particles comprise some or all of said ethylcellulose polymer, and wherein said aqueous medium comprises some or all of said water.

The following is a brief description of the drawings.

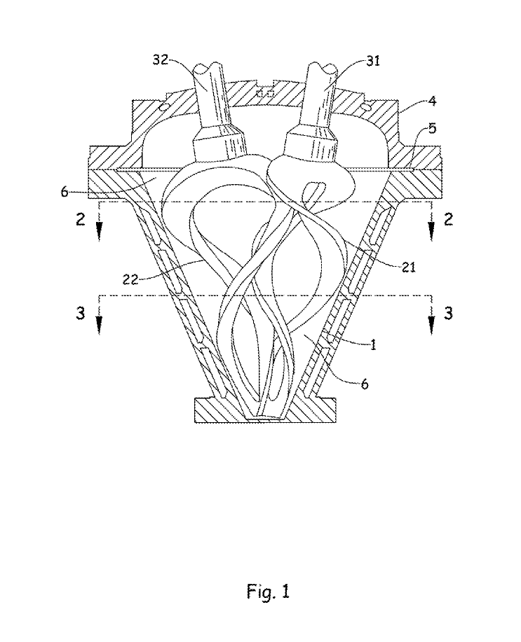

FIG. 1 is a vertical cross section of a mixer useful in one embodiment of the present invention.

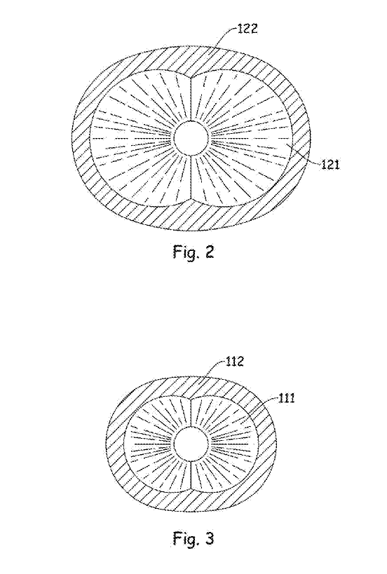

FIG. 2 is a horizontal cross section of the lower casing of the mixer shown in FIG. 1.

FIG. 3 is also a horizontal cross section of the lower casing of the mixer shown in FIG. 1.

The following is a detailed description of the invention.

As used herein, the following terms have the designated definitions, unless the context clearly indicates otherwise.

As used herein, an aqueous composition has 20% or more water by weight based on the weight of the composition. As used herein, a dispersion is a composition that contains a continuous medium that is liquid at 25° C. and contains discrete particles (herein called the “dispersed particles”) of a substance that are distributed throughout the continuous liquid medium. As used herein, an aqueous dispersion is an aqueous composition that is a dispersion in which the continuous liquid medium contains 50% or more water by weight based on the weight of the continuous liquid medium. Substances that are dissolved in the continuous liquid medium are considered herein to be part of the continuous liquid medium. The collection of all the dispersed particles is known herein as the “solid phase” of the dispersion.

As used herein, the “solids content” of an aqueous composition is the amount of material that remains when water and compounds having a boiling point of 250° C. or less have been removed. Solids content is characterized either by weight percent based on the total weight of the aqueous composition or by volume fraction based on the total volume of the aqueous composition.

Ethylcellulose polymer, as used herein, means a derivative of cellulose in which some of the hydroxyl groups on the repeating glucose units are converted into ethyl ether groups. The number of ethyl ether groups can vary. The USP monograph requirement for ethyl ether content is from 44 to 51%.

As used herein, the viscosity of an ethylcellulose polymer is the viscosity of a 5 weight percent solution of that ethylcellulose polymer in a solvent, based on the weight of the solution. The solvent is a mixture of 80% toluene and 20% ethanol by weight. The viscosity of the solution is measured at 25° C. in an Ubbelohde viscometer.

As used herein, a fatty acid is a compound having a carboxyl group and a fatty group. A fatty group is a linear or branched chain of carbon atoms connected to each other that contains 8 or more carbon atoms. A hydrocarbon fatty group contains only carbon and hydrogen atoms.

As used herein, a plasticizer is a compound that is miscible with ethylcellulose polymer, and that, when mixed with ethylcellulose polymer, reduces the glass transition temperature of that ethylcellulose polymer.

A compound is considered herein to be water soluble if 2 grams or more of the compound will dissolve in 100 grams of water at 25° C. A compound is considered water soluble even if it is required to heat the water to a temperature higher than 25° C. in order to form the solution, as long as the solution of 2 grams or more of the compound in water is a stable solution at 25° C.

A “polymer,” as used herein is a relatively large molecule made up of the reaction products of smaller chemical repeat units. Polymers may have a single type of repeat unit (“homopolymers”) or they may have more than one type of repeat unit (“copolymers”). Copolymers may have the various types of repeat units arranged randomly, in sequence, in blocks, in other arrangements, or in any mixture or combination thereof. Polymers have weight-average molecular weight of 2,000 daltons or higher.

The softening point of a material is the temperature below which the material behaves as a solid and above which it begins to be capable of flow under mild to moderate stress. Softening point is measured by the ring and ball method according to ASTM E28-14.

As used herein, a base is a compound that has the ability to accept a proton to form the conjugate acid of that compound, and the conjugate acid of that compound has pKa of 7.5 or greater.

As used herein, a fatty acid is a compound having a carboxyl group and a fatty group. A fatty group is a linear or branched chain of carbon atoms connected to each other that contains 8 or more carbon atoms. A hydrocarbon fatty group contains only carbon and hydrogen atoms.

As used herein, a “multiparticulate” is a plurality of particles. Particles are solid at 25° C. Particles are spherical or nearly spherical. If a particle is not spherical, its diameter is taken herein to be the diameter of a sphere having the same volume.

A container is said herein to be “sealable” if the container may be opened to allow ingredients to be put into the container and then sealed so that if the ingredients inside the container reach a pressure of 0.55 MPa (80 psig) or less, the container will not leak.

When it is stated herein that a ratio is X:1 or larger, it is meant that the ratio is Y:1, where Y is equal to or larger than X. For example, if it is stated that a certain ratio is 0.2:1 or larger, the ratio may be 0.2:1 or 0.5:1 or 100:1, but the ratio is not 0.1:1 or 0.02:1. Similarly, when is stated herein that a ratio is W:1 or smaller, it is meant that the ratio is Z:1, where Z is equal to or smaller than W. For example, if it is stated that a certain ratio is 5:1 or smaller, the ratio may be 5:1 or 4:1 or 0.1:1, but the ratio is not 6:1 or 10:1.

Any ethylcellulose polymer may be used in the present invention. The ethyl ether content of the ethylcellulose polymer is 44% or more; preferably 47% or more; more preferably 48% or more. The ethyl ether content of the ethylcellulose polymer is 51% or less; preferably 50% or less.

The ethylcellulose polymer preferably has viscosity of 2 mPa-s or higher; more preferably 5 mPa-s or higher; more preferably 12 mPa-s or higher; more preferably 16 mPa-s or higher. The ethylcellulose polymer preferably has viscosity of 120 mPa-s or lower; more preferably 100 mPa-s or lower; more preferably 80 mPa-s or lower; more preferably 60 mPa-s or lower; more preferably 40 mPa-s or lower; more preferably 30 mPa-s or lower.

The ethylcellulose preferably has softening point of 120° C. or higher; more preferably 130° C. or higher. The ethylcellulose preferably has softening point of 160° C. or lower; more preferably 150° C. or lower; more preferably 140° C. or lower.

Commercially available forms of ethylcellulose polymer which may be used in the invention include, for example, those available under the name ETHOCEL™, from The Dow Chemical Company. The ethylcellulose polymers used in the inventive examples are commercially available from The Dow Chemical Company as ETHOCEL™ Standard 4, ETHOCEL™ Standard 7, ETHOCEL™ Standard 10, ETHOCEL™ Standard 20, ETHOCEL™ Standard 45, or ETHOCEL™ Standard 100 with ethyl ether content from 48.0 to 49.5%. Other commercially available ethylcellulose polymers useful in embodiments of the invention include certain grades of AQUALON™ ETHYLCELLULOSE, available from Ashland, Inc., and certain grades of ASHACEL™ ethylcellulose polymers, available from Asha Cellulose Pvt. Ltd.

The present invention involves an aqueous dispersion. Preferably, the continuous liquid medium contains water in the amount, by weight based on the weight of the continuous liquid medium, of 60% or more; more preferably 70% or more; more preferably 80% or more; more preferably 90% or more.

Preferably, the dispersed particles in the aqueous dispersion contain ethylcellulose polymer in an amount, by weight based on the total dry weight of the solid phase, of 40% or more; more preferably 50% or more; more preferably 60% or more. Preferably, the dispersed particles in the aqueous dispersion contain ethylcellulose polymer in an amount, by weight based on the total dry weight of the solid phase, of 90% or less; more preferably 80% or less. A dispersed particle is considered herein to contain both material located on the interior of the particle and material located on the surface of the particle, such as, for example, a dispersant.

The composition of the present invention contains one or more fatty acids, which may be saturated or unsaturated. More preferred are unsaturated fatty acids. The fatty group of the fatty acid may be linear or branched; preferred is linear. The fatty group of the fatty acid may be a hydrocarbon fatty group or may have one or more substituent other than hydrogen or carbon; preferred are hydrocarbon fatty groups. Among unsaturated fatty acids, preferred are myristoleic acid, palmitoleic acid, sapienic acid, oleic acid, linoleic acid, and arachidonic acid. Among saturated fatty acids, preferred are caprylic acid, capric acid, lauric acid, palmitic acid, myristic acid, stearic acid, and arachidic acid. Most preferred is oleic acid.

Preferably the amount of fatty acid is, by weight based on the total dry weight of the solid phase, 2% or more; more preferably 4% or more; more preferably 6% or more. Preferably the amount of fatty acid is, by weight based on the total dry weight of the solid phase, 20% or less; more preferably 18% or less; more preferably 12% or less.

The composition of the present invention contains one or more base. The base is water-soluble. Preferred bases are ammonia, organic amines, alkali metal hydroxides, and alkaline earth hydroxides. The more preferred bases are ammonia and alkali metal hydroxides. The most preferred bases are alkali metal hydroxides. Non-fugitive bases are preferred over fugitive bases. Among fugitive bases, preferred are ammonia and fugitive bases that are organic amines. Among fugitive bases, more preferred is ammonia. Among organic amine fugitive bases, preferred are morpholine, alcohol amines, and mixtures thereof; more preferred are ammonia, morpholine, diethanolamine, 2-amino-2-methyl-1-propanol, and mixtures thereof.

Preferably the base:acid equivalent ratio of base to fatty acid is 1:1 or more; more preferably 1.1:1 or more. Preferably the base:acid equivalent ratio of base to fatty acid is 10:1 or less. When the base consists of one or more non-fugitive base, preferably the base:acid equivalent ratio of base compound to fatty acid is 2:1 or less; more preferably 1.5:1 or less. When the base consists of one or more fugitive base, preferably the base:acid equivalent ratio of base to fatty acid is 5:1 or more; more preferably 6:1 or more; more preferably 7:1 or more. When the base consists of a mixture of one or more fugitive base with one or more non-fugitive base, it is preferred that the base:acid equivalent ratio of the aggregate of all fugitive bases to fatty acid follows the preferred values described above for the base:acid equivalent ratio in the case where the base consists of one or more fugitive base, and it is preferred that the base:acid equivalent ratio of the aggregate of all non-fugitive bases to fatty acid follows the preferred values described above for the base:acid equivalent ratio in the case where the base consists of one or more non-fugitive base.

The method of the present invention involves the use of a mixer. The mixer is a device that includes a sealable volume. That is, part of the device is a container into which various ingredients may be put, and the container may be sealed. The size of the sealable volume is preferably 10 mL or more; more preferably 50 mL or more. The size of the sealable volume is preferably 1,000 L or less.

The mixer also includes one or more rotor. A rotor is a mechanical element that rotates around an axis. The rotor rotates within the sealable volume. Preferably, the mixer includes two or more rotors. Preferably, each rotor is driven by a drive shaft that passes through the casing of the mixer. Preferably, the casing of the mixer has a port through which the drive shaft can pass; preferably, the port allows the drive shaft to be driven by a motor located outside of the mixer while maintaining the seal that prevents leaking when the ingredients inside the sealable volume are at elevated pressure.

Rotors may have any shape or design. Some suitable rotors are cylindrical or conical and are rotated around the axis of the cylinder or cone. Among cylindrical or conical rotors, preferred are those having one or more helical grooves present on the surface. Examples of cylindrical or conical rotors are screws for extruders, either single-screw or twin-screw, and the paddles used in internal mixers designed to study polymer melt behavior.

A preferred shape for a rotor is the shape of a ribbon in a cylindrical or conical helix. That is, the ribbon is a helical shape that conforms to the surface of an imaginary cylinder or an imaginary cone. The rotor rotates around the axis of the imaginary cylinder or cone. Preferred is the conical helix. More preferred is a mixer having two opposing conical helix rotors that intermesh and that rotate in opposite directions.

Also contemplated are rotors in the shape of one or more impeller fixed to a shaft that rotates around the axis of the shaft. Examples of mixers using rotors in the shape of impellers are made by Parr instrument Company.

When material is in the mixer and the rotors are rotating, it is useful to characterize the volume of the material that is “contacted” by one or more of said rotors. For this characterization “material” refers to liquid and solid compounds or mixtures that are present in the mixer. An instant of time is chosen (the “analysis instant”) during step (e), and the space occupied by the material (labeled SM0) is considered. SM0 is the space occupied by the material and does not include the space occupied by any rotor. Because some or all of the rotor or rotors may be surrounded by material, the shape of SM0 may be extremely complex; SM0 may have voids corresponding to the location at which the rotor or rotors exist at the analysis instant. For purposes of characterizing the contacted volume, the shape and size of SM0 is considered to remain unchanging. Then, as the rotors rotate, some point on a rotor comes into contact with some point in SM0, and such a point is considered to be contacted. The volume of the space SM0 is VM0. After the rotors have completed one or more cycles of rotation, all of the contacted points can be observed, and the total volume of all such contacted points is the contacted volume (labeled VCON). The uncontacted volume is

VUNC=VM0−VCON.

The uncontacted volume, expressed as a percentage of VM0, is

% VUNC=100×VUNC/VM0

% VUNC is preferably 90% or less; more preferably 80% or less; more preferably 70% or less; more preferably 60% or less; more preferably 50% or less; more preferably 40% or less; more preferably 30% or less.

Preferably, as the rotor or rotors rotate, some portion of one or more rotor passes close to the interior surface of the container that contains the sealable volume. The distance of the closest approach made by any portion of any rotor to the interior surface of the container is the “clearance” of the mixer. Preferably, the clearance is 5 cm or less; more preferably 2 cm or less; more preferably 1 cm or less.

It is useful to characterize the proportion of the interior surface of the mixer that is approached closely by some point of a rotor. The interior surface of interest is the interior surface of the mixer that is in contact with the space SM0; that surface of interest is labeled Surf0. If, at any time during the rotation of a rotor, a point of the rotor approaches a point on Surf0 at a distance of double the clearance or less, that point on Surf0 is said herein to be approached closely by the rotor. Preferably, the area percentage of Surf0 made of closely approached points is 10% or more; more preferably 20% or more; more preferably 50% or more, based on the area of Surf0.

Preferably, the mixer is equipped with apparatus for heating the mixer. Preferably, heat is applied to the exterior of the mixer in a way that allows the heat to transfer to the materials in the sealable volume. For example, heated fluid may circulate through a jacket on the exterior of the mixer in a way that allows heat to transfer through the wall of the mixer to the materials in the sealable volume.

Preferably, the mixer is equipped with apparatus that allows a gas to be injected into the sealable volume while the sealable volume is sealed, to raise the pressure inside the sealed sealable volume. Preferred gasses to be injected are inert gasses, including nitrogen and the noble gasses. More preferred is nitrogen. Preferably, the mixer is equipped with apparatus that allows liquid to be injected into the sealable volume while it is sealed, without losing the seal. Preferred liquids to be injected are water and solutions of water soluble base dissolved in water.

FIG. 1 shows a vertical cross section of a mixer suitable for one embodiment of the present invention. The mixer may be separated into two parts: the bowl 1 and the cap 4. Material is placed into the bowl 1, and the bowl 1 and the cap 4 are brought together at seal 5. One rotor has blade 21 and drive shaft 31; the other rotor has blade 22 and drive shaft 32. The drive shafts pass through cap 4 without compromising the ability of the mixer to maintain pressure. The blades 21 and 22 leave open volume 6 that is partially or fully occupied by ingredients. Drive shafts 31 and 32 are driven in rotation by one or more motors (not shown) that are mechanically coupled to drive shafts 31 and 32. The cap 4 also has apparatus (not shown) that allows liquid to be inserted into the open volume 6 without compromising the ability of the mixer to maintain pressure. The cap 4 also has apparatus (not shown) that allows gas to be inserted into the open volume 6 without compromising the ability of the mixer to maintain pressure. The cap 4 also has apparatus (not shown) that allows measurements of conditions inside the mixer (for example, temperature and pressure) to be conveyed (for example, via an electrical signal on one or more wires) to the exterior of the mixer without compromising the ability of the mixer to maintain pressure.

FIG. 2 and FIG. 3 are horizontal cross sections of the bowl 1. FIG. 2 shows the wall 122 of the bowl and the inside surface 121 inside the bowl. FIG. 3 shows the wall 112 of the bowl and the inside surface 111 of the cavity inside the bowl.

In the practice of the present invention, materials are placed into the mixer. The materials include ethylcellulose polymer, fatty acid, water, and base. After some preliminary steps, ethylcellulose polymer, fatty acid, water, and base are in the sealable volume of the mixer, the sealable volume is sealed. Then the mixture of the materials is subjected to a temperature that is above the softening point of the ethylcellulose polymer at the same time that the rotors are rotating.

In some embodiments (“one-shot” embodiments), materials including ethylcellulose polymer, fatty acid, water, water-soluble base, and optional additional ingredients are placed in the mixer. As the materials are placed in the mixer, the mixer may be at any temperature from 15° C. to 99° C. Steps (b) and (c) may be performed in any order or simultaneously, with the ethylcellulose polymer, fatty acid, water, water-soluble base, and optional additional ingredients added in any order. At some convenient time, the rotors are brought into contact with the materials. At some time after the materials are in the sealable volume, the sealable volume is sealed. At some convenient time before or after the sealable volume is sealed, the materials are heated to a temperature above the softening point of the ethylcellulose polymer, and the rotation of the rotors is begun. The rotation of the rotors may be begun at any convenient time before or after the sealable volume is sealed. The rotation of the rotors at the temperature above the softening point of the ethylcellulose polymer is continued after the sealable volume is sealed until the composition containing dispersed particles of ethylcellulose polymer (as described below) is formed.

In other embodiments (“two shot” embodiments), materials including ethylcellulose polymer, fatty acid, and optional additional ingredients are placed in the mixer. As the materials are placed in the mixer, the mixer may be at any temperature from 15° C. to 99° C. Then the ethylcellulose polymer, fatty acid, and optional additional ingredients are heated to a temperature high enough to make the material mixture soft enough to allow the rotors to rotate, and the rotors are rotated. Up until this point, the sealable volume may or may not have been sealed. Then the sealable volume is sealed if it was not already sealed. Then the following steps are taken, in any order that is convenient: a solution of water-soluble base in water is injected into the sealed sealable volume (that is, step (c) is performed); the materials in the sealable volume are heated to a temperature above the softening point of the ethylcellulose polymer, and the rotation of the rotors is begun. The rotation of the rotors at the temperature above the softening point of the ethylcellulose polymer is continued after the sealable volume is sealed until the composition containing dispersed particles of ethylcellulose polymer (as described below) is formed.

Regardless of whether a one-shot embodiment or a two-shot embodiment or some other embodiment is used, a preferred procedure is to conduct the method so that a concentrated dispersion is first produced. A concentrated dispersion is composition that contains dispersed particles in which the solid phase constitutes 70% or more by weight of the composition, based on the total weight of the composition. It is preferred that while the ethylcellulose polymer, fatty acid, water, water-soluble base, and optional additional ingredients are all present in the mixer, and the rotors are being rotated while the materials are at a temperature above the softening point of the ethylcellulose polymer, the amounts of ethylcellulose polymer, fatty acid, water, water-soluble base, and optional additional ingredients have been chosen so that the amount of water is 30% or less by weight based on the total weight of the materials in the mixer. More preferably the amount of water, by weight based on the total weight of the materials in the mixer, is 26% or less; more preferably 23% or less.

Preferably, when a concentrated dispersion is made, after step (e) (as defined above) is performed, additional water (labeled “dilution water”) is added to the composition. Preferably, after addition of the dilution water, the composition in the mixer is still a composition of the present invention, and the solids level is, by weight based on the weight of the composition, 5% or higher; more preferably 10% or higher. Preferably, after addition of the dilution water, the composition in the mixer is still a composition of the present invention, and the solids level is, by weight based on the weight of the composition, 40% or lower; more preferably 30% or lower.

Preferably, after the composition containing dispersed particles of ethylcellulose polymer is made, the composition is removed from the mixer.

The method of the present invention is a batch process. That is, various ingredients are put into the mixer, the method of the present invention is completed, an amount of the aqueous composition is produced, and that amount of aqueous composition is removed from the mixer prior to placing any further ingredients into the mixer. The present invention does not include methods in which some but not all of the composition containing dispersed particles of ethylcellulose particles is removed from the mixer, after which further ethylcellulose polymer is placed into the mixer.

The aqueous composition of the present invention preferably has pH of 12 or lower; more preferably 11 or lower; more preferably 10 or lower. The aqueous composition of the present invention has pH of 8 or higher.

The dispersed particles in the aqueous composition of the present invention preferably have volume-average particle diameter of 3 micrometers or less; more preferably 2 micrometer or less. The dispersed particles in the aqueous composition of the present invention preferably have volume-average particle diameter of 50 nm or greater; more preferably 100 nm or greater. Particle size was measured using laser diffraction. A suitable instrument is a COULTER™ LS-230 or COULTER™ LS-13-320 particle size analyzer (Beckman Coulter Corporation).

The viscosity of the aqueous composition of the present invention is measured at 25° C. using a Brookfield RV-II viscometer with an RV2 or RV3 spindle spinning at 50 rpm. The spindle is chosen to give the torque signal nearest to the center of the viscometer's torque range. Preferably the viscosity of the aqueous composition is 100 mPa-s or lower; more preferably 80 mPa-s or lower; more preferably 60 mPa-s or lower; more preferably 40 mPa-s or lower; more preferably 30 mPa-s or lower. Preferably the viscosity of the aqueous composition is 1 mPa-s or higher.

The aqueous composition of the present invention preferably has a solids content, by weight based on the weight of the aqueous composition, of 5% or more; more preferably 10% or more; more preferably 15% or more; more preferably 20% or more. The aqueous composition of the present invention preferably has a solids content, by weight based on the weight of the aqueous composition, of 55% or less; more preferably 50% or less; more preferably 45% or less; more preferably 40% or less; more preferably 35% or less.

A preferred use for the aqueous composition of the present invention is to produce a film. The aqueous composition of the present invention is optionally mixed with additional ingredients; a layer of the aqueous composition of the present invention is applied to a surface, and the water is removed. The resulting film preferably contains residual water in an amount, by weight based on the weight of the film, of 0 to 5%; more preferably 0 to 2%; more preferably 0 to 1%; more preferably 0 to 0.5%.

The resulting film may be used for any purpose. A preferred purpose is as a pharmaceutical coating or a food coating; more preferred is a pharmaceutical coating; more preferred is a modified-release pharmaceutical coating. A preferred method of making a modified-release pharmaceutical coating is to provide a multiparticulate formulation that contains a drug and apply a coating of the film to envelop or encapsulate each of the multiparticulates. Preferred multiparticulates are made from sugar or microcrystalline cellulose and have a drug applied as a layer to the surface or sprayed onto the surface. Alternatively, multiparticulates may contain a drug located in the interior of the particles, for example if the multiparticulates are made by extrusion followed by spheronization of a mixture of the drug with the material that will be made into the multiparticulates. The coating formed by the film made from the aqueous composition of the present invention preferably forms a complete layer of coating on 50% or more of the particles (by number); more preferably, the coating forms a complete layer of coating on 75% or more of the particles (by number). Preferably, on 90% or more of the particles (by number), the coating covers 75% or more of the area of the surface of each particle.

Suitable multiparticles may be pellets, granules, powders, or other forms.

Also contemplated are embodiments in which the film is used as a modified-release coating on pharmaceutical dosage forms such as tablets or capsules.

When an aqueous composition of the present invention is used for making a film, it is preferred to use a plasticizer.

When a plasticizer is used, the plasticizer may be added to the composition at any point during the process of making the composition. Preferably, when a plasticizer is used, the plasticizer is added at the same time as the ethylcellulose polymer (that is, during step (b). Also envisioned are embodiments in which a composition of the present invention is made and then plasticizer is added, either in the same mixer or in some other vessel after the composition has been removed from the mixer in which it was made.

When a plasticizer is used, preferred are one or more plasticizers selected from the group consisting of triglycerides, organic esters, polyethylene glycol of molecular weight of 200 or higher, and alkyl carboxylic acids; more preferred are triethyl citrate (TEC), dibutyl sebacate (DBS), diethyl phthalate, dibutyl phthalate, polyethylene glycol of molecular weight of 200 or higher, and triglycerides; more preferred are triethyl citrate (TEC), dibutyl sebacate (DBS), diethyl phthalate, and dibutyl phthalate.

When a plasticizer is used, the amount of plasticizer preferably is, by weight based on the total dry weight of the solid phase, 10% or more; more preferably 15% or more. When a plasticizer is used, the amount of plasticizer preferably is, by weight based on the total dry weight of the solid phase, 40% or less; more preferably 30% or less.

When a composition forms a coating on a plurality of particles, it is desired that the coating have good film properties, such as relatively high values of Young's modulus, tensile strength, and maximum elongation. It is contemplated that these properties may be tested by making a free film (that is, a film that is not attached to any substrate) and testing the tensile properties of the free film. It is contemplated that films that have acceptable properties as free films will also have acceptable properties when coated onto multiparticulates.

The following are examples of the present invention.

Vmean is the volume-average particle diameter. “D<90%” is the diameter below which 90% by volume of the particles fall. Particle size “mode” is the diameter at which the peak in the curve of particle population versus diameter is observed. Particle diameters are measured using COULTER™ LS-230 or COULTER™ LS-13-320 particle size analyzer (Beckman Coulter Corporation).

### EXAMPLE 1

### One-Shot Embodiment With Low Molecular Weight Ethylcellulose Polymer and KOH

Mixer: Mixer as depicted in FIG. 1. Size of the Sealable volume was 1.9 liter (4 pints).

Proportions by Weight: (74/17/9) 74 parts Ethocel Std. 10; 17 parts dibutyl sebecate; 9 parts oleic acid. Base was KOH.

The heater was initially set to 90° C. The mixer was charged with 196.1 g ETHOCEL™ Std. 10 powder, 45.05 g dibutyl sebecate (DBS), 23.85 g oleic acid, 20.85 g 30% wt KOH, 62 ml DI (deionized) water. The blades were turned on at a low rate after the bowl was sealed up during the initial water addition to completely wet the powder. The bowl was then pressurized to 0.517 MPa (75 psig) with nitrogen, and the bath set point was increased to 165° C. This temperature set point eventually resulted in a measured bowl temperature of 145° C. During the heat up, the mixers were turning slowly and once the temperature leveled off the mixer was turned on at max rate for 30 minutes. After this initial mixing the dilution water was added at 5 ml/min for the first 200 ml, then 15 ml/min for the remaining 483 ml to give a batch that was 26.5% solids by loaded ingredients. The mixer remained on at max rate during the dilution.

After all the water was added the mixing was stopped and the heater set point was dropped back down to 90° C. Once the measured bowl temperature dropped below 100° C. the pressure was bled off and the material in the bowl was recovered, analytical given below.

The solids loaded into the bowl were almost completely converted into a light grey dispersion (965 g material recovered, 1000 g loaded).

30.75% solids, pH=8.73, particle size mode=106 nm

### EXAMPLE 2:

### 2-Shot Process With High Molecular Weight Ethylcellulose Polymer and KOH

Same mixer and proportions as in Example 1.

The heater was initially set to 90° C. and charged with 196.1 g ETHOCEL™ Std. 20 powder, 45.05 g dibutyl sebecate (DBS), 23.85 g oleic acid. The mixer was then sealed, pressurized to 0.517 MPa (75 psig) with nitrogen, and heated to a set point of 165° C., which gave a measured bowl temperature of 145° C. At this point 20.85 g 23% wt KOH (base:acid equivalent ratio of 1.2:1) and 62 ml deionized (DI) water were added while the mixer was turning slowly. Once all the water and base were added the mixer was turned on at maximum rate (75 rpm) for 30 minutes. After this initial mixing the dilution water was added at 5 ml/min for the first 200 ml, then 15 ml a min for the remaining 305 ml to give a batch that was approximately 38% solids by weight based on the loaded ingredients. The mixer remained on at maximum rate during the dilution.

After all the water was added the mixing was stopped and the heater set point was dropped back down to 90° C. Once the measured bowl temperature dropped below 100° C. the pressure was bled off and the material in the bowl was recovered, analytical given below. There was a small amount of solid material present upon emptying of the mixer. The particle size distribution had a main peak at approximately 100 nm and small amount of larger size material in the 4-10 micrometer range.

Analytical results: 38.5% solids, pH=8.83, particle size mode=106 nm, Vmean=0.704 micrometer, D<90%=0.210 micrometer.

### EXAMPLE 3

### 2-Shot Process With High Molecular Weight Ethylcellulose Polymer and Ammonia

Same mixer and same proportions as in Example 1. Base was ammonia.

The heater was initially set to 90° C. and charged with 196.1 g ETHOCEL™ Std. 20 powder, 45.05 g dibutyl sebecate (DBS) and 23.85 g oleic acid. The mixer was sealed and pressurized to 0.517 MPa (75 psig) and the bath temperature was set to 165° C., which eventually gave a measured bowl temperature of 145 ° C. At this point the mixer was turned on to a slow speed, the bath set point was lowered to 155° C. and 46.21 ml 28% ammonia solution (base:acid equivalent ratio of 8:1) and 44.9 ml water (22% water by weight) were delivered into the bowl under slow mixing with syringe pumps. After the water and base were added the mixer was set to its maximum speed and run for 30 minutes. After this initial mixing the dilution water was added at 5 ml/min for the first 200 ml. After this first dose of dilution water there was considerable foaming in the mixing bowl. Next, 494 ml dilution water was added at 15 ml/min to give a batch that was 26.5% solids target by loaded ingredients. The mixer remained on at max rate during the dilution.

After all the water was added the mixing was stopped and the heater set point was dropped back down to 90° C. Once the measured bowl temperature dropped below 100° C. the pressure was bled off and the material in the bowl was recovered.

The material in the bowl was mostly aqueous dispersion, with some crumbly solid and some foam. 837 g of material was recovered out of 1034 g loaded (80.9% recovery). This analytical for this dispersion is given below:

pH=9.45, solids=14.72%, Vmean=35.39 (1 peak at 200 nm, multiple peaks between 2-200 micrometers)

### EXAMPLE 4

### 1-Shot Process With High Molecular Weight Ethylcellulose Polymer With Ammonia

Same mixer and proportions as in Example 1. Base was ammonia

The heater was initially set to 90° C. and charged with 196.1 g ETHOCEL™ Std. 20 powder, 45.05 g dibutyl sebecate (DBS), 23.85 g oleic acid, 46.21 g of 28% ammonia solution in water, and 44.9 ml of DI water. The mixer was then sealed, pressurized to 0.517 MPa (75 psig) with nitrogen, and mixed slowly for a couple of minutes to wet the dry ingredients. The bowl heater was turned on to a set point of 165° C., which gave a measured bowl temperature of 145° C. Once this temperature was reached the mixer was turned on at max rate (75 rpm) for 30 minutes. After this initial mixing the dilution water was added at 5 ml/min for the first 350 ml, then 15 ml a min for the remaining 250 ml to give a batch that was 27.7% solids by weight by loaded ingredients. The mixer remained on at max rate during the dilution.

After all the water was added the mixing was stopped and the heater set point was dropped back down to 90° C. Once the measured bowl temperature dropped below 100° C. the pressure was blend off and the material in the bowl was recovered, analytical given below. There was a considerable amount of foaming during the last few psi of depressurization, likely from dissolved excess ammonia. The particle size distribution had a main peak at approximately 350 nm and small amount of larger size material in the 2-50 micrometer range.

Analytical results were as follows: solids=28.5%, pH=8.71, particle size mode=358 nm, Vmean=4.18 micrometer, D<90% =10.32 micrometer.

### EXAMPLE 5

### 2-Shot Process With Low Molecular Weight Ethylcellulose Polymer, With Ammonia (4:1)

Same mixer and proportions as Example 1. Base was ammonia.

The heater was initially set to 90° C. and charged with 196.1 g Ethocel Std. 10 powder, 45.05 g dibutyl sebecate (DBS) and 23.85 g oleic acid. The mixer was sealed and pressurized to 0.517 MPa (75 psig) and the bath temperature was set to 165° C., which eventually gave a measured bowl temperature of 145° C. At this point the mixer was turned on to a slow speed, the bath set point was lowered to 155° C. and 23.1 ml 28% ammonia solution (base:acid equivalence ratio of 4:1) and 59.85 ml water were delivered into the bowl with Isco syringe pumps. After the water and base were added the mixer was set to its maximum speed and run for 40 minutes. After this initial mixing the dilution water was added at 5 ml/min for the first 200 ml, then 15 ml a min for the remaining 460 ml to give a batch that was 26.5% solids by weight by loaded ingredients. The mixer remained on at max rate during the dilution.

After all the water was added the mixing was stopped and the heater set point was dropped back down to 90° C. Once the measured bowl temperature dropped below 100° C. the pressure was bled off and the material in the bowl was recovered.

The recovered material was not a dispersion of polymer particles in water. The recovered material was a soft crumbly solid with a slight amount of free water.

### EXAMPLE 6

### 1-Shot Process With Low Molecular Weight Ethylcellulose Polymer With Ammonia (3.5:1)

Same mixer and proportions as in Example 1. Base was ammonia.

The heater was initially set to 90° C. and charged with 196.1 g ETHOCEL™ Std. 10 powder, 45.05 g dibutyl sebecate (DBS), 23.85 g oleic acid, 20.22 g 28% wt ammonia (base:acid equivalent ratio 3.5:1), and 62 ml DI water. The blades were turned on at a low rate after the bowl was sealed up during the initial water addition to completely wet the powder. The bowl was then pressurized to 0.517 MPa (75 psig) with nitrogen, and the bath set point was increased to 165° C. This temperature set point eventually resulted in a measured bowl temperature of 145° C. During the heat up, the mixers were turning slowly, and once the temperature leveled off, the mixer was turned on at max rate for 30 minutes. After this initial mixing the dilution water was added at 5 ml/min for the first 200 ml, then 15 ml/min for the remaining 468 ml to give a batch that was 26.5% solids by weight by loaded ingredients. The mixer remained on at max rate during the dilution.

After all the water was added the mixing was stopped and the heater set point was dropped back down to 90° C. Once the measured bowl temperature dropped below 100° C. the pressure was bled off and the material in the bowl was recovered.

The recovered material was not a dispersion of polymer in water. The material in the bowl was completely converted into a soft crumbly solid with a slight amount of free water. Upon standing overnight this free water was absorbed into the solid, which became harder when cool.

### EXAMPLE 7

### 1-Shot Process With High Molecular Weight Ethylcellulose Polymer and Ammonia (2.5:1).

Same mixer and proportions as Example 1. Base was ammonia.

The heater was initially set to 90° C. and charged with 196.1 g ETHOCEL™ Std. 20 powder, 45.05 g dibutyl sebecate (DBS), 23.85 g oleic acid, 14.55 g 28% ammonia solution (base:acid equivalent ratio of 2.5:1), 57.8 ml DI water. The blades were turned on at a low rate after the bowl was sealed up during the initial water addition to completely wet the powder. The bowl was then pressurized to 0.517 MPa (75 psig) with nitrogen, and the bath set point was increased to 175° C. This temperature set point eventually resulted in a measured bowl temperature of 155° C., at which point the mixer was turned on at max rate for 30 minutes. After this initial mixing the dilution water was added at 10 ml/min for the first 300 ml, then 15 ml/min for the remaining 375 ml to give a batch that was 26.5% solids by loaded ingredients.

After all the water was added the mixing was stopped and the heater set point was dropped back down to 90° C. Once the measured bowl temperature dropped below 100° C. the pressure was blend off and the material in the bowl was recovered.

The recovered material was grey powdery solid and a large amount of dark brown water. From the % solids measurement (0.4%) of this water we can tell that very little of the loaded solids were in the water phase.

### EXAMPLE 8

### Parr Mixer, Low Molecular Weight Ethylcellulose Polymer, KOH

Same proportions as Example 1. Base was KOH.

Reaction vessel was a 300 mL Parr vessel (model 4560) reactor fitted with a Cowles blade. The Cowles blade has an approximate 2″ diameter. The Parr vessel was initially charged with 47.60 g Ethocel Std. 10 powder, 11.02 g dibutyl sebecate (DBS), 5.81 g oleic acid, 5.10 g 30% wt KOH, 16.18 ml DI water. The vessel was then sealed up, the heater temperature was set to 145° C., and the Cowles mixing blade was turned on to a low speed. No external pressure was applied to the reactor. During the heat up, the mixer was turning slowly and once the temperature leveled off the mixer was turned on at max rate (approximately 1800 rpm) for 30 minutes. After this initial mixing the dilution water was added with a high performance liquid chromatograph (HPLC) pump at 5 ml/min for the first 47.5 ml, then 15 ml/min for the remaining 120 ml to give a batch that was 26.05% solids by weight by loaded ingredients. The mixer remained on at max rate during the dilution.

After all the water was added, the heating mantle was dropped to allow the vessel to cool while still mixing at max rate. Once the measured vessel temperature dropped below 50° C. the material in the vessel was recovered, analytical given below. The solids loaded into the bowl were completely converted into a light grey dispersion. The measured % solids is 24.56% which is slightly lower than the theoretical solids.

### COMPARATIVE EXAMPLE C-EX (DISPERSION MADE IN AN EXTRUDER)

Extrusion is a continuous process and does not fall within the present invention.

The comparative extruder based ETHOCEL dispersion was prepared using the following components and conditions:

Component 1: ETHOCEL Std. 20

Component 1 feed rate: 42.0 g/min

Component 2: Dibutyl sebecate

Component 2 feed rate: 9.6 g/min

Component 3: Oleic acid

Component 3 feed rate: 5.1 g/min

Initial Water feed rate: 14.6 g/min

Base: 28 wt. % ammonia solution in water

Base feed rate: 1.9 g/min

Dilution Water feed rate: 140 g/min

Extruder Temperature in Polymer Melt Zone: 145° C.

Extruder Speed: 470 rpm

The general procedure was as follows.

Component 1 was fed into a 25 millimeter (mm) diameter twin screw extruder using a controlled rate feeder; using the feed rate in grams/minute (g/min) as indicated above. Components 2 and 3 were fed into a liquid injector in the melt zone of the extruder and combined with component 1 to form a liquid melt material.

The extruder temperature profile was ramped up to approximately 145° C. Water and base were mixed together and fed to the extruder at a rate indicated above for neutralization at an initial water introduction site. Then dilution water was fed into the dilution zone of the extruder be a controlled rate pump at the rate indicated above. The extruder speed was approximately 470 revolutions per minute (rpm). At the extruder outlet, a backpressure regulator was used to adjust the pressure inside the extruder barrel to a pressure adapted to reduce steam formation (generally, the pressure is about 2 Mpa (about 300 psia)).

The particle size of the solids particles of the aqueous dispersion was measured using a Coulter LS-230 particle size analyzer (available from Beckman Coulter Corporation).

### EXAMPLE 9

### Results From Selected Examples

| Ex | Mixer | Base | ECP | base amt. | shots | Mode | Vmean | D < 90% |
|---|---|---|---|---|---|---|---|---|
| 1 | rib | KOH | low | 1.4:1 | 1 | 0.106 μm | 0.491 μm | 0.214 μm |
| 2 | rib | KOH | high | 1.4:1 | 2 | 0.106 μm | 0.704 μm | 0.210 μm |
| 3 | rib | Amm | high | 8:1 | 2 | 98.5 μm | 35.4 μm | 122 μm |
| 4 | rib | Amm | high | 8:1 | 1 | 0.358 μm | 4.185 μm | 10.32 μm |
| 5 | rib | Amm | low | 4:1 | 2 | No disp. | No disp. | No disp. |
| 6 | rib | Amm | low | 3.5:1 | 1 | No disp. | No disp. | No disp. |
| 7 | rib | Amm | high | 2.5:1 | 1 | No disp. | No disp. | No disp. |
| 8 | imp | KOH | high | 1.4:1 | 1 | 0.117 μm | 37.44 μm | 140 μm |
| C-Ex. | extr | Amm | high | 1.8:1 | cont | 0.141 μm | 0.144 μm | 0.230 μm |

Ex = Example number
Amm = ammonia
ECP = ethylcellulose polymer
low = ETHOCEL ™ Std 10
high = ETHOCEL ™ Std 20
base amt. = base:acid equivalent ratio
cont = continuous process
mode = particle size mode, in micrometers
Vmean = volume-average particle size in micrometers
D < 90% = diameter below which 90% of the particles reside, by volume
No disp. = no dispersion was formed
rib = ribbon mixer depicted in FIG. 1
imp = Parr reactor with impeller
extr = extruder

The method of the present invention is capable of producing a dispersion using either a ribbon mixer or an impeller mixer; the ribbon mixer generally produced dispersions of smaller particle size. KOH produced dispersions at much lower molar amount of base. Good dispersions were produced with both low and high molecular weight ethylcellulose polymer. Among the ammonia samples, better dispersions were produced at higher amounts of base.

### COMPARATIVE EXAMPLE C-A: FOUR-PIN MIXER

Four-pin mixers are described, for example, in WO 2008/052112. A four-pin mixer is a device in which four cylindrical rods, parallel or slightly tipped, arranged in a square, are inserted into a container along with ingredients to be mixed, and the rods are each rotated around its own axis. When the rods rotate, they do not encounter any point in space other than the volume of the rod itself. Thus the rotation of the rod does not cause the rod to encounter any points in space formerly occupied by the material being mixed. Thus the % VUNC for a four-pin mixer is 100%.

The four-pin mixer that was tested was not heated, so the effectiveness of the four-pin mixer was assessed using silicone oil and surfactant instead of ethylcellulose and fatty acid, tested at ambient temperature (approximately 23° C.). Silicone oil was either silicone oil having viscosity of 100,000 mPa-s (100,000 cps) or silicone oil having viscosity of 300,000 mPa-s (300,000 cps). Surfactant was a mixture of NEODOL™ 23-65 ethoxylate from Shell with water, in varying proportions. Weight ratio of silicone oil to surfactant/water mixture was 94:6. The pins were rotated according to two different schemes: in one scheme, all the rods rotate in the same direction; in the other scheme, adjacent rods rotate in opposite directions. Mixtures were processed for either 5 minutes or 10 minutes. A concentrated dispersion was made in the four-pin mixer; the concentrated dispersion was then diluted, and the particle size was analyzed using a Coulter Counter.

Among the various formulations and process variable combinations using the four-pin mixer, several resulted in stable dispersions, but the smallest particle size produced by any of them had volume-average particle diameter of 2 micrometer.

### COMPARATIVE EXAMPLE C-B: SILICONE OIL IN THE MIXER OF FIG. 1.

Silicone oil formulations as described in Comparative Example C-A were processed in the same mixer used in Example 1. Ingredients were as follows:

Oil 100=silicone oil having viscosity of 100 mPa-s

Oil 300=silicone oil having viscosity of 300 mPa-s

Surf-1=NEODOL™ 23-65 surfactant from Shell

Surf-2=EMPIRICOL™ ESB 70 surfactant from Huntsman Two different mixing procedures were used. In the “pre-mix” procedure, the water and surfactant were mixed together prior to placing them into the mixer along with the silicone oil (Denoted in the table below by putting surfactant and water in parenthesis). In the “no-pre-mix” procedure, silicone oil, surfactant, and water were all brought together in the mixer. The amounts of materials (parts by weight) and the resulting particle size of the dispersion are shown in the table below. Particle size is the volume-average diameter.

| Mixture | Mixing Conditions | Size (μm) |
|---|---|---|
| 90 Oil-1/6 Surf-1/4 water | 5 min at 43 rpm | 2.1 |
| 90 Oil-1/6 Surf-1/4 water | 10 min at 120 rpm | 0.7 |
| 95 Oil-1/3 Surf-1/2 water | 5 min at 43 rpm | 1.4 |
| 95 Oil-1/3 Surf-1/2 water | 10 min at 120 rpm | 0.8 |
| 90 Oil-1/10 (Surf-1/water 60:40) | 5 min at 43 rpm | 4.0 |
| 90 Oil-1/10 (Surf-1/water 60:40) | 10 min at 120 rpm | 1.6 |
| 95 Oil-1/5 (Surf-1/water 60:40) | 5 min at 43 rpm | 1.9 |
| 95 Oil-1/5 (Surf-1/water 60:40) | 10 min at 120 rpm | 0.8 |
| 94.8 Oil-2/5.8 Surf-2/3.5 water | 10 min at 120 rpm | 1.8 |

The results of Comparative Examples C-A and C-B show the superiority of the twin-ribbon mixer used in C-B over the four-pin mixer used in C-A. Nearly all the dispersions produced in C-B had smaller particle size than the very smallest result (2 micrometers) produced by C-A. It is contemplated that if the same two mixers were used for the production of ethylcellulose polymer dispersions, the twin-ribbon mixer would again produce smaller particle size than the four-pin mixer.

1. A method of making an aqueous composition comprising
(a) providing a mixer comprising a sealable volume and one or more rotors inside said sealable volume;
(b) placing into said sealable volume ingredients comprising ethylcellulose polymer and fatty acid;
(c) placing into said sealable volume ingredients comprising water and a water-soluble base;
(d) after said steps (b) and (c), sealing said sealable volume or allowing said sealable volume to remain sealed;
(e) then rotating one or more of said rotors while said ingredients are at a temperature above the softening point of said ethylcellulose polymer, to produce said aqueous composition;
wherein said step (e) is conducted so that 90% or less of the volume of said ingredients is uncontacted by one or more of said rotors;
wherein said aqueous composition comprises particles dispersed in an aqueous medium, wherein said particles comprise some or all of said ethylcellulose polymer, and wherein said aqueous medium comprises some or all of said water.

2. The method of claim 1, wherein said steps (b) and (c) are performed prior to sealing said sealable volume.

3. The method of claim 1, wherein said step (b) is performed to form a mixture comprising said ethylcellulose polymer and said fatty acid, and wherein said mixture is maintained at a temperature above the softening point of said ethylcellulose polymer prior to performing said step (c).

4. The method of claim 1, wherein said water soluble base consists of one or more fugitive base, and wherein the equivalent ratio of said water soluble base to said fatty acid is 5:1 or more.

5. The method of claim 1, wherein said water soluble base consists of one or more non-fugitive base, and wherein the equivalent ratio of said water soluble base to said fatty acid is 1:1 or more, and wherein the equivalent ratio of said water soluble base to said fatty acid is 2:1 or less.
